(19) United States
(12) Patent Application Publication
(10) Pub. No.: US 2024/0083970 A1
(43) Pub. Date: Mar. 14, 2024

(54) COMPOSITIONS COMPRISING FUSION PROTEIN AND ANALYTICAL ATTRIBUTES THEREOF

(71) Applicant: DR. REDDY’S LABORATORIES LIMITED, Hyderabad (IN)

(72) Inventors: Korampalli SAIDARAO, Guntur (district) (IN); Haribabu AAMUDALAPALLI, Hyderabad (IN); Sharath Chandra RAGHUPROLU, Nagar Nagarkurnool (District) (IN); Pandiaraja PANDURANGAN, Lawspet (Post) (IN); Suranjana HALDAR, Kolkata (IN); Samir Kumar MANDAL, Hyderabad (IN)

(21) Appl. No.: 18/275,242

(22) PCT Filed: Feb. 1, 2022
(86) PCT No.: PCT/IN2022/050083

§ 371 Date: Aug. 1, 2023

(30) Foreign Application Priority Data
Feb. 1, 2021 (IN) 202141004301

(51) Int. Cl. C07K 14/705

(52) U.S. Cl. CPC C07K 14/70521; C07K 2319/30

(57) ABSTRACT

The present invention discloses a therapeutic composition comprising fusion protein with reduced heterogeneity in the glycosylation profile of the protein and methods thereof. More particularly the invention provides the composition with reduced heterogeneity in the glycosylation profile. By reducing heterogeneity, the resultant preparation is expected to exhibit superior, consistent results in terms of safety, purity and potency. The invention is of particular importance as it can form the part of the critical quality attributes (CQA) that help in ensuring batch-to-batch consistency and predicted shelf-life of complex protein molecules.

### FIELD OF INVENTION

The present invention relates to compositions comprising fusion proteins and analytical attributes thereof. More particularly, the invention provides glycosylation profile of compositions comprising Fc-fusion protein.

### BACKGROUND OF THE INVENTION

Post-translational modifications (PTMs) play a key role in influencing the physiological function of a given protein. While the activity of therapeutic glycoproteins is mainly determined by the amino acid sequence; the pharmacokinetics, solubility, stability and enhancement of receptor function are primarily the influence of the PTMs. For this reason, mammalian cell lines are a preferred expression system, so that a desired PTM profile is achieved. Specifically, glycosylation has been reported to affect the efficacy, stability, immunogenicity, clearance rate, antibody-dependent cellular cytoxicity (ADCC), and complement-dependent cytoxicity (CDC), among others.

The resultant glycosylation profile in a given glycoprotein composition is however not only influenced by the aforementioned in vivo factors (i.e., the specific cell line of production and the intracellular glycosylation machinery) but also by in vitro factors such as cell culture conditions and the multiple levels of upstream and downstream processing that the protein is subject to. All these factors can lead to the microheterogeneity (in glycan structure) and/or macroheterogeneity (in glycan profile viz., variablilty in occupancy of the possible glycosyaltion sites in the protein sequence) of the preparation which can affect the immunogenicity, effector functions and the pharmacokinetic properties of the drug and consequently, the final drug quality. Thus, there is a considerable motivation and challenge to monitor, characterize and, if required, modulate the glycosylation profile throughout every different developmental stage.

CTLA4-Ig fusion protein is a glycoprotein that contains N- and O-glycosylation sites resulting in structurally complex glycans. For example, abatacept (marketed as Orencia® by Bristol-Myers Squibb) is a cytotoxic T-lymphocyte-associated antigen-Immunoglobulin G1 fragment fusion protein (CTLA4-Ig). Abatacept is a glycosylated homodimeric protein (monomers linked by a single inter-chain disulphide bond) with each 357 amino acid long monomer reported to bear three N-linked (Asn76, Asn108 in the CTLA-4 region and Asn207 in the Fc region) and two O-linked (Ser129 and Ser139) glycosylation sites. Compared to monoclonal antibodies, abatacept shows a higher level of structural complexity due to the higher number of glycosylation sites. Further, these glycan species are highly branched (especially the N-linked species) and terminally sialylated (in both N- and O-linked glycans).

This adds to the complexity in the development of a fusion protein preparation that is expected to demonstrate reduced heterogeneity such that acceptable and consistent standards of quality, biological activity, safety, and efficacy are achieved.

### SUMMARY OF THE INVENTION

Accordingly, the present invention discloses a composition comprising a fusion protein with reduced heterogeneity in the glycosylation profile and related analytical methods. The post-translational modification (PTM) profile of a therapeutic protein preparation, which also comprises the glycosylation profile, has a considerable bearing on its stability, safety and efficacy. By reducing heterogeneity, the resultant preparation is expected to exhibit superior consistency in terms of safety, purity and potency. The invention is of particular importance as it can form part of the critical quality attributes (CQA) that help in ensuring batch-to-batch consistency and predicted shelf-life of complex protein molecules.

### BRIEF DESCRIPTION OF DRAWINGS

FIG. 1. Schematic representation of the primary protein structure of a CTLA-4-IgG fusion protein which exists as dimer of identical monomeric polypeptide chains. Each monomeric polypeptide chain consists of 357 amino acids, of which residues 1 to 125 forms the extra cellular CTLA-4 domain, while residues 126 to 357 forms IgG HC Fc domain.

FIG. 2. Representative profile of abatacept N-glycan chromatogram showing start and end time points.

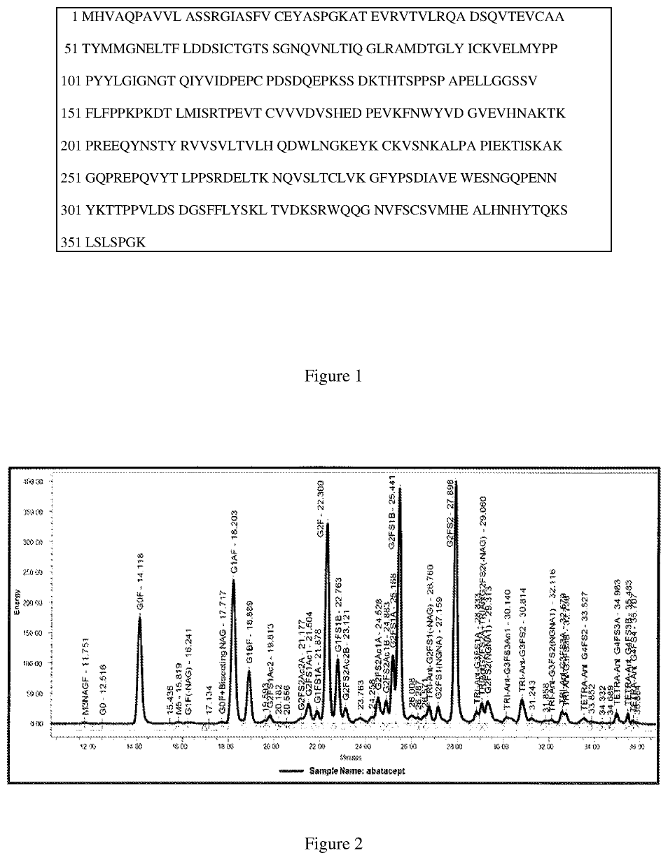

### DETAILED DESCRIPTION OF THE INVENTION

### Definitions

The term “about” refers to a range of values that are similar to the stated reference value to a range of values that fall within 25, 20, 19, 18, 17, 16, 15, 14, 13, 12, 11, 10, 9, 8, 7, 6, 5, 4, 3, 2, 1 percent above or below the stated reference value.

The term “glycoprotein” or “biotherapeutic” used herein interchangeably refers in the broadest sense and it covers proteins that are genetically engineered through recombinant DNA technology, which are of therapeutic significance in the treatment of ailments. Biotherapeutics include monoclonal antibodies, fusion proteins, polyclonal antibodies, multispecific antibodies and antibody fragments so long as they exhibit the desired biological activity.

The term “composition” or “preparation” used herein refers to a population of biotherapeutic molecules that is produced by mammalian cell culture. The population may have one or several post translational modifications (PTM), imparting the molecules a different molecular weight, charge, solubility or combinations thereof.

The term “fusion protein composition” refers to a population of fusion protein molecules or fragments thereof that is produced by mammalian cell culture. The population of fusion protein molecules may have one or several post translational modifications (PTM), imparting the fusion protein molecules a different molecular weight, charge, solubility or combinations thereof.

The term “galactosylated species” used herein refers to the total mono- and di-galactosylated species in the preparation. The glycoprotein composition encompassed by the present invention is represented in Table 1.

The term “glycan” refers to monosaccharide or polysaccharide moiety attached to another molecule. The term “glycoprotein” refers to any polypeptide or protein which has one or more covalently attached glycan. The term “glycan variant” as used herein refers to a glycoprotein subunit in the composition with a given glycan species.

The term “glycoform” used herein refers to one or more of different molecular variants of a glycoprotein resulting due to variable glycan attachment site occupancy on the glycoprotein.

The term “glycosylation profile” as used herein refers to the profile of glycoprotein composition in terms of individual glycoforms and/or their amounts present in that composition. The amounts of the individual glycoform in the profile can be absolute numerical values or a range of numerical values.

The term “heterogeneity” used herein refers to a phenomenon of existence of diverse glycoforms in a glycoprotein preparation wherein, an increase in diversity corresponds to increase in heterogeneity. Heterogeneity can cover both macro- and micro-heterogeneity wherein, the former refers to site occupancy of glycan moieties, while microheterogeneity refers to variations in the glycan structure at a specific site.

The term “high mannose species” as used herein refers to N-linked glycans that contain unsubstituted terminal mannose sugars. These glycans typically contain between five and nine residues attached to the chitobiose (NAG2) core. The number associated with the abbreviation as given in table 1 indicates the number of mannose residues in the structure.

The term “multiantennary species” used herein refers to the total tri- and tetra-antennary glycans wherein three and four NAG sequences are added to the glycan core structure in total tri- and tetra-antennary glycans respectively. The glycoprotein composition encompassed by the present invention is represented in Table 1. The term “N-glycan” or “N-linked glycan” as used herein refers to the N-linked glycosylated glycans in which glycans are attached to the asparagine of the recognition sequence Asn-X-Thr/Ser, where “X” is any amino acid except proline.

The term “O-glycan” or “O-linked glycan” as used herein refers to O-linked glycosylated glycans in which glycan is attached to oxygen atom of serine or threonine residues of the protein.

The term “post-translational modification” or “PTM”, herein used interchangeably, refers to biochemical modification that occurs at one or more amino acids on a protein molecule after translation of the protein. PTMs are mostly chemical or enzyme-mediated, at specific target sequences in the protein and comprise inter alia, glycosylation, glycation, acetylation, amidation, deamidation, methylation, ADP-ribosylation and hydroxylation.

The term “sialylated N-glycan” as used herein refer to the N-glycan species which have sialic acid in terminal positions. This may include mono-, bi-, and/or tri-antennary glycans with mono, di, tri and tetra sialylated glycans. The glycoprotein composition encompassed by the present invention may have one or more sialylated N-Glycan species is represented in Table 1.

The term “target glycosylation profile” as used herein refers to predetermined, characteristic glycosylation profile of glycoprotein composition in terms of individual glycoforms and/or their amounts present in that composition. For example “target sialylation profile” of a glycoprotein would refer to a predetermined, characteristic sialylation profile of the glycoprotein in terms of individual sialylated glycoforms/and or their amounts present in that glycoform composition. These target glycosylation profile can be based on existing monographs for that glycoprotein, approved specification for the glycoprotein by regulatory agencies, or a quality control criterion developed for pharmaceutical preparation of that glycoprotein. The amounts of the individual glycoform in this target glycosylation profile can be absolute numerical values or a range of numerical values.

The present invention discloses a composition comprising CTLA-4-IgG fusion protein with glycosylation profile of the protein and methods thereof.

A person of ordinary skill in the art would be able to determine the glycosylation profile of the composition described herein using the state-of-the-art techniques. (Ruhaak, L. R., et al., Glycan 15 labeling strategies and their use in identification and quantification. Anal Bioanal Chem, 2010. 397(8): p. 3457-81; Wuhrer, M., A. M. Deelder, and C. H. Hokke, Protein glycosylation analysis by liquid chromatography-mass spectrometry. J Chromatogr B Analyt Technol Biomed Life Sci, 2005. 825(2): p. 124-33; Guile, G. R., et al., A rapid high-resolution high-performance liquid chromatographic 20 method for separating glycan mixtures and analyzing oligosaccharide profiles. Anal Biochem, 1996. 240(2): p. 210-26). Unless defined otherwise, all technical and scientific terms used herein have the same meaning as commonly understood by a person having ordinary skill in the art to which the invention pertains.

In an embodiment, the invention discloses a composition comprising a fusion protein comprising N-glycan variants. In an embodiment, the N-glycan variants are selected from a list comprising high mannose species, total galactosylated species, total sialylated species, di- & tri-sialylated species and total multiantennary species. In another embodiment, the invention discloses a composition comprising a fusion protein comprising O-glycan variants. In another embodiment, the O-glycan variants are selected from a list comprising monosialylated species, disialylated species, multi-sialylated species, total sialylated species, total asialylated species and maltose adducts. In a yet another embodiment, the invention discloses a composition comprising a fusion protein comprising N-glycan variants and O-glycan variants.

In an embodiment, the invention discloses a composition comprising a fusion protein comprising N-glycan variants wherein the composition comprises one or more of about 0.19% to about 0.22% high mannose species; about 58.9% to about 65.6% total galactosylated species; about 48.3% to about 57.0% total sialylated species, about 20.6% to about 26.4% di- & tri-sialylated species, and about 8.4% to about 10.2% total multiantennary species.

In another embodiment, the N-linked glycosylation is at one or more of Asn76, Asn108, and Asn207. In another embodiment, the O-linked glycosylation is at one or more of Ser129, Ser 130, Ser 136, Ser139, and Ser148.

In yet another embodiment, the composition belongs to a given batch of production.

In a further embodiment, the fusion protein is a CTLA-4-IgG fusion protein.

In yet another embodiment, the CTLA-4-IgG fusion protein is abatacept.

In another embodiment, the invention discloses a method of producing a composition comprising a fusion protein wherein, the method comprises:
a) analytical testing of one or more of N-linked glycosylation profile and O-linked glycosylation profile;
b) acquiring values for one or more of the parameters listed in Table 1,
c) assessing whether the values of step b) fall within a target glycosylation profile and
d) tailoring manufacturing processes such that the values of step b) fall within the target glycosylation profile, thereby producing the therapeutic composition.

In yet another embodiment, the composition belongs to a given batch of production.

In yet another embodiment, the manufacturing process comprises upstream manufacturing process or downstream manufacturing process.

In a further embodiment, the fusion protein is a CTLA-4-IgG fusion protein.

In yet another embodiment, the CTLA-4-IgG fusion protein is abatacept.

### Abbreviations

ACN: Acetonitrile
CHO: Chinese hamster ovary
CQA: Critical quality attributes
DTT: Dithiothreitol
F: Fucose
GOF: asialo-agalacto-fucosylated biantennary glycan
Ig: Immunoglobulin
M: Mannose
NAG: N-acetyl glucosamine
NGNA: N-glycolylneuraminic acid
PTM: post-translational modification
S: Sialic acid
SEC: Size exclusion chromatography

### EXAMPLES

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

TABLE 1
Representative N-Glycan species

| Species name | Formula |
|---|---|
| High mannose | M5 |
| Species | |
| Total Galactose | GlAF, GlBF, GlF-(NAG), G2F, G2FS1(NGNA), |
| | G2FS1A, G2FS1-Acl, G2FS1-Ac2, G2FS1B, Tetra- |
| | Ant-G4FS2, Tetra-Ant-G4FS3A, Tetra-Ant-G4FS3B, |
| | Tri-Ant- G2FS1(-NAG), Tri-Ant-G3FS1A, Tri-Ant- |
| | G3FS2, Tri- Ant-G3FS2(NGNA1) |
| Total Sialylated | G1FS1A, G1FS1B, G2FS1A, G2FS1-Acl, |
| | G2FS1Ac2, G2FS1B, G2FS2, G2FS2(NGNA1), |
| | G2FS2-Ac1A, G2FS2-Ac1B, G2FS2-Ac2A, G2FS2- |
| | Ac2B, Tetra-Ant-G4FS2, Tetra-Ant-G4FS3A, Tetra- |
| | Ant-G4FS3B, Tetra-Ant-G4FS4, TriAnt-G2FS1 |
| | (-NAG), Tri-Ant-G3FSlA, Tri-Ant-G3FS2, Tri-Ant- |
| | G3FS2(NGNA1), Tri-Ant-G3FS3A, Tri-Ant-G3FS3- |
| | Ac1, Tri-Ant-G3FS3B, [Tri-Ant-G2FS2A + Tri-Ant- |
| | G2FS2(-NAG)], G2FS1(NGNA) |
| Di + tri Sialylated | G2FS2, G2FS2(NGNA1), G2FS2-Ac1A, G2FS2- |
| | Ac1B, G2FS2-Ac2A, G2FS2-Ac2B, Tetra-Ant- |
| | G4FS2, Tetra-Ant-G4FS3A, Tetra-Ant-G4FS3B, Tri- |
| | Ant-G3FS2, Tri-Ant-G3FS2(NGNA1), Tri-Ant- |
| | G3FS3A, Tri-Ant-G3FS3-Ac1, Tri-Ant-G3FS3B, |
| | [Tri-Ant-G2FS2A + Tri-Ant-G2FS2(-NAG)] |

### Example I

Glycoproteins in the preparation was deglycosylated using PNGaseF in deglycosylation buffer containing 5% RapiGest™ (w/v). The released N-Glycosylamines are labelled using GlycoWorks RapiFluor-MS reagent solution. For this, RapiFluor—MS reagent solution was added to the above deglycosylated mixture. After incubation at room temperature, samples were diluted with 100% Acetonitrile (ACN). Labelled N-Glycosylamines were captured on a GlycoWorks HILIC SPE μElution™ plate and eluted using 200 mM Ammonium acetate (pH 7) in 5% ACN. The eluted mix was diluted in dimethylformamide (DMF)/CAN solution and mixed thoroughly. Samples were then analysed by HPLC (ACQUITY UPLC BEH Glycan Separation Technology Column). All the individual glycan species were integrated as shown in FIG. 2 and peak annotation of each species were followed based on elution order for identification as per the retention time.

### Example II

One or more of the N-glycan species as in Table 1 of a test preparation may be compared with a given standard preparation described below.

Analytical measurement of N-glycan species in the composition is first done using methodology as described in Example I. Based on the percentage of each species thus obtained, a positive or negative (+/−) score is given against each row corresponding to a given glycan species, for whether the value corresponding to the relative abundance of a species falls within the target glycosylation profile. The target glycosylation profile (indicating target ranges of glycan species) of the abatacept composition of present invention is shown in Table 2. The test preparation that scores as positive for the set of N-glycan species under test at a given time is selected for further suitability procedures for commercial release. For example, Table 3 indicates the corresponding scores of two hypothetical test preparations X and Y. A to H indicate a given set of N-glycan species under consideration for qualification of the commercial batch. Test preparation X scores positive for all parameters checked, whereas test preparation Y does not fall within the acceptable ranges of A, E and F. Thus test preparation Y may be rejected in the qualification step. Further, manufacturing processes adopted for test preparation Y may be altered to generate samples which will then be subject to subsequent qualification.

TABLE 2
Target glycosylation profile of abatacept

| Sl. No. | N-glycan species | Percentage range |
|---|---|---|
| 1 | High mannose species | about 0.19% to about |
| | | 0.22% |
| 2 | total galactosylated species | about 58.9% to about |
| | | 65.6% |
| 3 | total sialylated species | about 48.3% to about |
| | | 57.0% |
| 4 | di- & tri-sialylated species | about 20.6% to about |
| | | 26.4% |
| 5 | total multiantennary | about 8.4% to about |
| | species | 10.2% |

TABLE 3
A representative qualification method for commercial release

| N-glycan | Score for Test | Score for Test |
|---|---|---|
| species type | Preparation X | Preparation Y |
| A | + | − |
| B | + | + |
| C | + | + |
| D | + | + |
| E | + | − |
| F | + | − |
| G | + | + |
| H | + | + |

The invention may be embodied in other specific forms without departing from the spirit or essential characteristics thereof. The foregoing embodiments and examples are therefore to be considered in all respects illustrative rather than limiting the invention described herein.

1. A fusion protein composition from a batch of production comprising N-glycan variants wherein the composition comprises one or more of:
about 0.19% to about 0.22% high mannose species; about 58,9% to about 65.6% total galactosylated species; about 48.3% to about 57.0% total sialylated species, about 20.6% to about 26.4% di- & tri-sialylated species, and about 8.4% to about 102% total multiantennary species.

2. The composition as claimed in claim 1 wherein the N-glycan variants bear N-linked glycosylation at one or more of Asn76, Asn108, and Asn207 of the fusion protein.

3. A method of producing a CTLA4-IgG fusion protein composition wherein, the method comprises:
a) analytical testing of N-linked glycosylation profile;
b) acquiring values for one or more of the parameters listed in Table 1;
c) assessing whether the values of step b) falls within a target glycosylation profile and
d) tailoring manufacturing processes such that the values of step b) falls within the target glycosylation profile;
thereby producing the protein composition.

4. The method as claimed in claim 3 wherein, the manufacturing process comprises upstream manufacturing process or downstream manufacturing process.

5. The method as claimed in claim 3, wherein, the fusion protein is a CTLA-4-IgG fusion protein.

6. The method as claimed in claim 5, wherein, the CTLA-4-IgG fusion protein is abatacept.

7. The composition as claimed in claim 1, wherein, the fusion protein is a CTLA-4-IgG fusion protein.

8. The composition as claimed in claim 7, wherein, the CTLA-4-IgG fusion protein is abatacept.
